(19) United States
(12) Patent Application Publication
(10) Pub. No.: US 2022/0378117 A1
(43) Pub. Date: Dec. 1, 2022

(54) Adaptive Wrap Top

(71) Applicant: Alina Kuderska, Brooklyn, NY (US)

(72) Inventor: Alina Kuderska, Brooklyn, NY (US)

(21) Appl. No.: 17/334,578

(22) Filed: May 28, 2021

(51) Int. Cl. A41C 3/02; A41C 3/00

(52) U.S. Cl. CPC A41C 3/02; A41C 3/0028

(57) ABSTRACT

A top garment, such as a wrap top, bra, or similar garment has a supportive back portion made to improve and maintain the wearer's posture. A front portion made to overlap a chest of the wearer. An interfaced elastic bottom panel, an elastic element that maintains the place of the garment, and a closure mechanism that allows the wearer to easily dress and remove the garment. The closure mechanism, additionally, allows for the wearer to adjust the pressure and control the compression the garment is applying to the torso.

### BACKGROUND INFORMATION

There are a number of different wrap top garments used by the general public today. A majority of these garments are difficult to put on and take off or lack adjustability of support. Prior tops were constructed with clip closing mechanism that were usually located on the back of a person making it difficult for persons with physical disabilities to dress and remove the top. Other tops required a person to lift the top over their head to dress and remove. This presents difficulties to individuals with low shoulder mobility, especially those in the elderly community. These tops suffered from a number of shortcomings in the adaptability to the elderly community. First, the lack of comfortability to dress and remove. Secondly, the lack of adjustability. The classic clip closing mechanism not only is difficult to maneuver especially for people with arthritis, it also limits the wearer's ability to adjust the compression the top applies to the upper torso. This makes current tops on the market particularly uncomfortable for individuals who spend a large portion of their day in a wheelchair. In addition, many tops include permanent breast pads which can make the top uncomfortable.

### SUMMARY

Some exemplary embodiments described herein, is a garment made up of a supportive layer on the back, a bottom elastic panel, a closure mechanism located on the front of the bottom elastic panel. There is a need for an adaptive top to provide support for those who are elderly and or sit for large portions of the day. The structure of the adaptive wrap top provides support and helps improve the posture of the wearer.

Various features and advantages will appear from the description to follow. In the description, reference is made to the accompanying drawings which form apart thereof, and in which is shown by way of illustration and for practicing this invention. This embodiment will be described in sufficient detail to enable those skilled in the art to practice the invention. It is to be understood that other embodiments maybe utilized and that structural changes may be made without departing from the scope of the invention.

### BRIEF DESCRIPTION OF THE DRAWINGS

FIG. 1 shows a back view of the adaptive wrap top in an open position according to various exemplary embodiments.

FIG. 2 shows a front elevational view of the adaptive wrap top shown in FIG. 1 as it would appear on a wearer.

FIG. 3 shows a side elevational view of the adaptive wrap top shown in FIG. 1 as it would appear on a wearer.

FIG. 4 shows a back elevational view of the adaptive wrap top shown in FIG. 1 as it would appear on a wearer.

FIG. 5 shows a front view of the adaptive wrap top in an open position according to various exemplary embodiments.

### DETAILED DESCRIPTION

The exemplary embodiments may be further understood with reference to the following description and the related appended drawings, wherein like elements are provided with the same reference numerals. The exemplary embodiments describe a garment, and more specifically an adaptive wrap top that surrounds the upper portion of a person's torso to provide support and improve posture. This garment is adapted to allow for easy application and removal.

The adaptive top of the exemplary embodiments is a garment that allows a person of little dexterity to apply the adaptive wrap top themselves. This is accomplished through an interfaced supportive bottom elastic panel and a front opening and closing mechanism, each of which will be described in greater detail below.

The adaptive wrap top of the exemplary embodiments is also designed to cover the chest while providing support to reduce back pain and improve the wearer's posture. The adaptive wrap top also offers an easy opening and closing mechanism that allows the wearer to easily adjust the pressure the adaptive wrap top applies to the torso of the wearer.

The exemplary embodiments may be particularly useful for women or men who are elderly, are constricted to a wheelchair, and/or have limited dexterity. However, the exemplary embodiments are not limited to the wearers being this type of person. The adaptive wrap top of the exemplary embodiments offers a supportive back layer that helps maintain and improve the posture of the wearer. Often in the elderly and disabled community, back issues arise from long hours being spent in wheelchairs and sitting down. This is due to nature of slouching or hunching over while sitting down which causes strain on the discs in the vertebrae. The adaptive wrap top of the exemplary embodiments helps to alleviate and prevent these injuries by inhibiting the wearer from sitting in a slouched or hunched position.

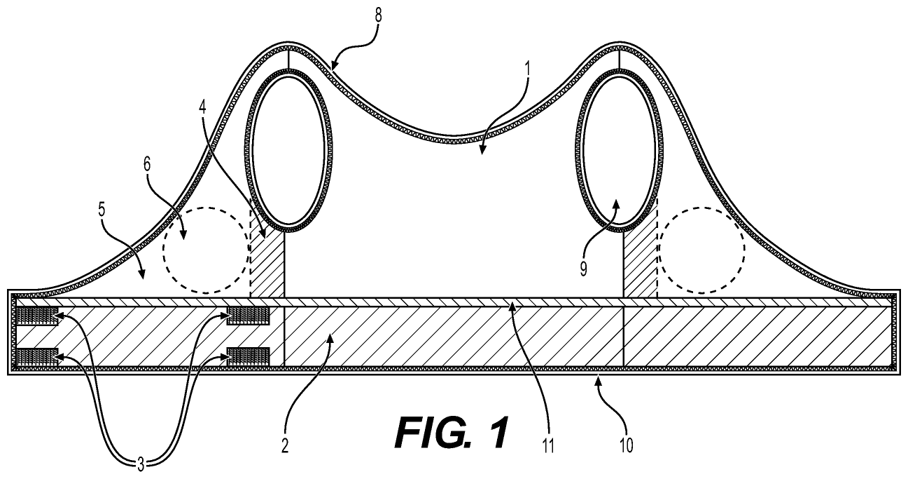

FIG. 1 shows a back view of the adaptive wrap top 10 in an open position according to various exemplary embodiments. FIG. 1 shows the entirety of the adaptive wrap top 10 laid out flat. The view of FIG. 1 is the adaptive wrap top 10 that is laid out flat when it is removed from a wearer. The surface that is facing the viewer is the surface that would be facing away from the wearer when the adaptive wrap top 10 is worn. The adaptive wrap top 10 includes a supportive back layer 1, an interfaced elastic bottom panel 2, closure mechanism 3, interfaced supportive side panels 4, a front portion 5, the removable bra pad 6, an elastic finishing and structuring hem 8, armholes 9, and anti-slip strip 11.

In FIG. 1, the supportive back portion 1 includes a doubled layer supportive material that helps to maintain posture. In some exemplary embodiments the supportive back layer portion 1 may be doubled layered to provide extra support. The additional layer may be placed in the form of cross and locked with a stitch. The materials used to make the supportive back portion may include, but are not limited to, cotton, elastic, polyester, linen, rayon, any types of blends, including recycled and any other fabrics out of natural vegetable and man-made fibers. A person who is skilled in the art would recognize the appropriate materials to use or be modified to be used for this supportive back portion. This supportive back portion 1 improves the posture of the wearer throughout the day and helps to reduce back pain. The interfaced elastic bottom panel 2 provides support around the body. The interfaced elastic bottom panel 2 is configured to provide a comfortable fit as the elastic panel can be tightened and loosened based on the wearer's preference. Interfacing is a technique used with fabric to make certain parts of a garment more stable. It is a common method known to those skill in the art that shape, structure, and support to the clothes. Commonly, it is an additional layer which is applied to the inside of garments to add firmness.

The interfaced supportive side panels 4 are configured to provide side structural support. These weaves or inserted interfaced supportive side panels 4 are placed adjacent to the removable bra pads 6. This helps the breast to stay in place and secures the breasts in their desirable location. The materials used to make the interfaced supportive side panels 4 may include but are not limited to plastic, cotton, cotton-polyester blends, and any other fabric that is made out of man-made fibers as well as vegetable natural fibers, including recycled materials, that adds supportive stiffened quality to the panel. A person who is skilled in the art would recognize the appropriate materials to use or be modified to be used for this supportive back portion. Some exemplary embodiments of the interfaced supportive side panels 4 include anti-slip strips or a coating to keep the adaptive wrap top 10 from rising up on the wearer's torso. The materials used to create the anti-slip strip and/or coating may include but are not limited to silicone and vinyl and other materials that provide anti-slip qualities. Moreover, the interfaced supportive side panels 4 are configured to keep the removable bra pads 6 from moving when placed in the adaptive wrap top 10.

The adaptive wrap top 10 also includes an elastic finishing that may include stretch lace trim and other trim. The adaptive wrap top 10 may also include a structuring hem 8 which is located around the edge of the adaptive wrap top 10 to provide contour and support the shape of the garment. Lastly, there are two armhole 9 provided through the supportive back portion 1 to allow the wearer to place both arms through, where some exemplary embodiments have elastic stretch lace trim finishing. This allows for the wearer place the garment on their back, wrap the adaptive wrap top 10 around the front of their torso, pull the fused elastic bottom panel on both sides to the middle of their torso, and lastly secure the garment closed with the hook and loop Velcro closing mechanism to the pressure of their preference. In other exemplary embodiments, wherein the closure mechanism may include a series of press stud fasteners or snaps, or hook and eye, instead of or in addition to the hook and loop closures.

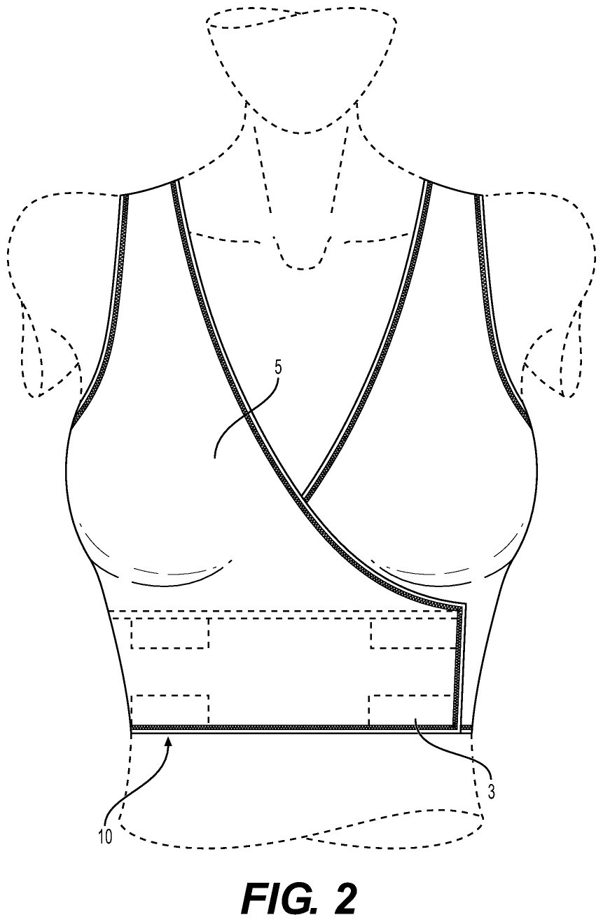

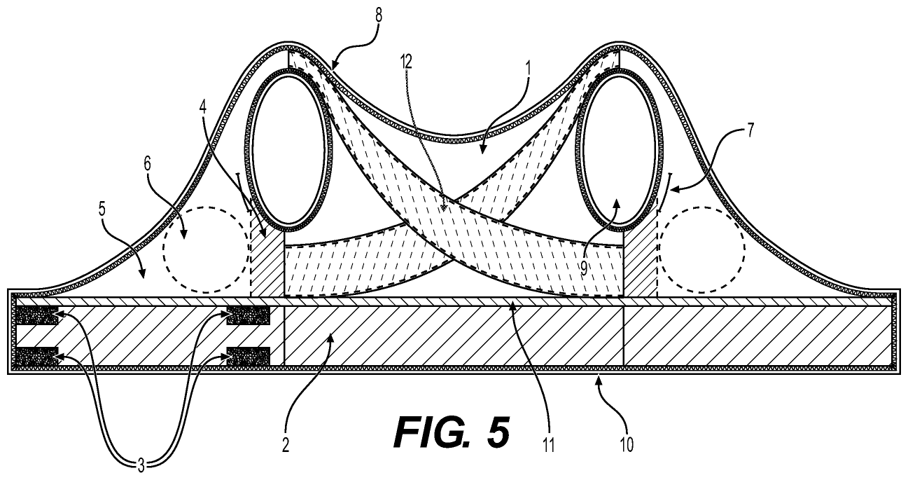

FIGS. 2, 3 and 4 show the front, side and back elevational drawing of the adaptive wrap top as it would appear on the wearer. Moreover, these figures show the outer portion of the garment that will face away from the wearer's body, whereas FIG. 5 shows the inside portion that will lie against the wearer's body.

FIG. 2 shows the front elevational drawings of the front portion 5 as it would appear on the wearer. Additionally, it shows the closure mechanism 3 in the closed position as it would appear on the wearer. This view shows the adaptive wrap 10 in the upper torso of the wearer's body where it is intended to be worn.

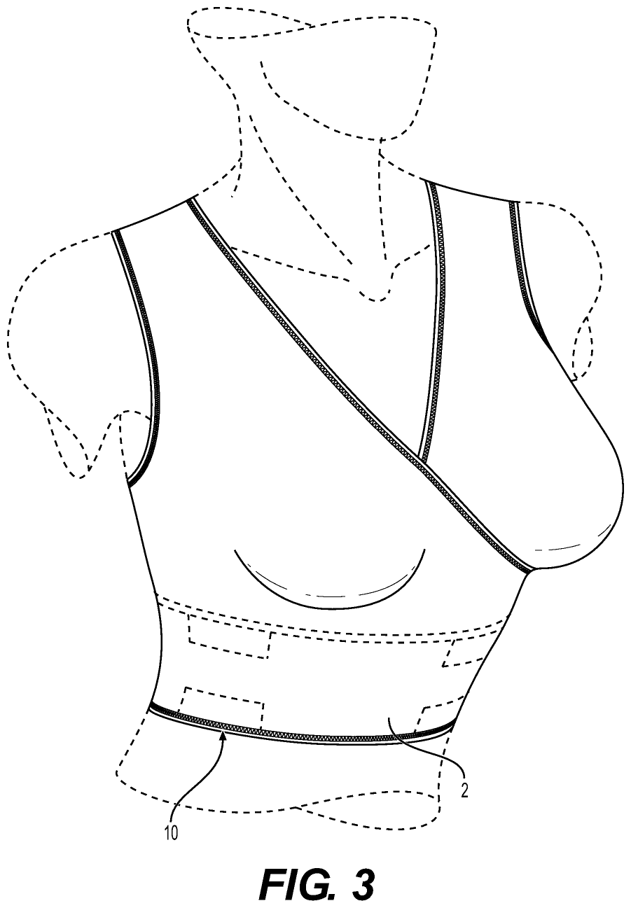

FIG. 3 shows the side elevational side elevational drawing of the adaptive wrap top 10 as it would appear on the wearer. This figure shows the interfaced elastic bottom panel 2 as it would appear on the wearer and the ideal location on the wearer's torso.

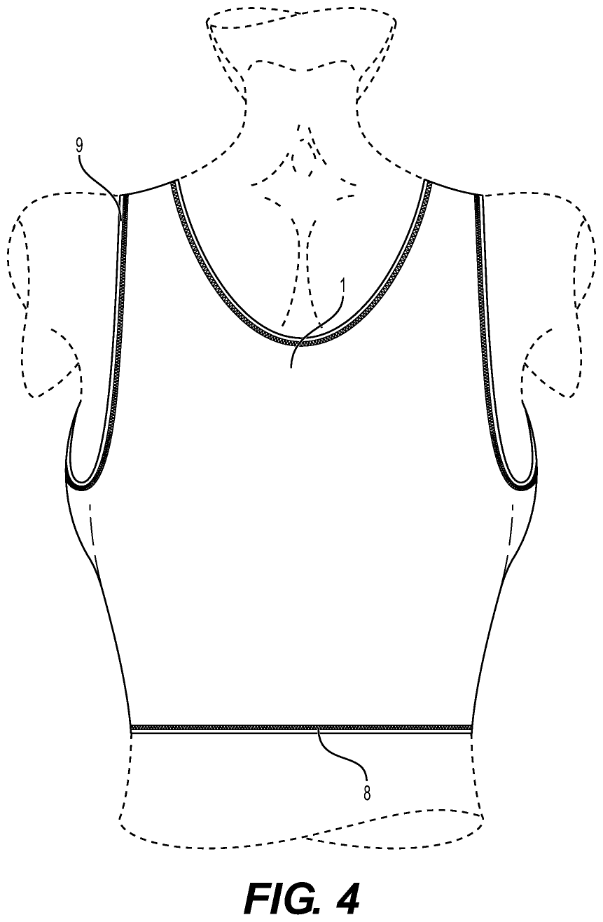

FIG. 4 shows the supportive back portion 1 as it would appear on a wearer. Furthermore, FIG. 4 shows how the wearer places their arms through the armholes 9. Lastly, FIG. 4 shows how the structing hem 8 contours the supportive back portion 1. As shown in FIG. 4, the supportive back portion 1 substantially covers the upper portion and the middle portion of the wearer's back. This allows the supportive back portion 1 to exerts a pressure against the upper and middle portion of the wearer's back when the adaptive wrap top 10 is being worn. This pressure provides support and improves the wearer's posture.

FIG. 5 shows a front view of the adaptive wrap top 10 in an open position according to various exemplary embodiments. The view of FIG. 5 is the adaptive wrap top 10 that is laid out flat when it is removed from a wearer. The surface that is facing the viewer is the surface that would be against the wearer's body when the adaptive wrap top 10 is worn. It shows the supportive back portion 1 with an additional cross supportive feature 12. This cross supportive feature 12 comprises of a secondary material interfaced from the upper right portion to the lower left portion, and a third material interfaced from the upper left portion to the lower right portion. This overlap of material forms a cross over the wearer's back and provides additional corrective support to improve the wearer's posture. The materials used to make the cross supportive feature may include, but is not limited to, cotton, elastic, polyester, linen, rayon, any types of blends, including recycled and any other fabrics out of natural vegetable and man-made fibers.

A series of hook and loop fastener closures 3 are stitched to the interfaced elastic bottom panel 2, that in some variants may include an anti-slip strip 11 to help garment stay in place, e.g., to keep the adaptive wrap top 10 from rising up on the wearer's torso. The materials used to create the anti-slip strip and/or coating may include but are not limited to silicone and vinyl and other materials that provide anti-slip qualities. In some exemplary embodiments, there are two hook and two loop fastener closures 3 stitched to the inside of the interfaced elastic bottom panel 2 and another set of two hook and two loop fastener closures 3 stitched to the outside of the interfaced elastic bottom panel 2. The hook and loop fastener closures 3 are configured to provide a smooth and soft closing mechanism that allows for hook and loop to merge and keep the adaptive wrap top closed without catching onto other clothing or uncomfortably rubbing the wearer.

Furthermore in FIG. 5, the adaptive wrap top 10 includes the removable bra pads 6 which are configured to provide support, smooth shape, and protection. The materials used to make the removable bra pads 6 may include, but are not limited to any cotton, foam, and silicone. The adaptive wrap top 10 has the two openings 7 for the bra pads 6. The openings 7 are sewn slits into pockets of the inner front portion 5 of the adaptive wrap top 10 to allow the bra pads 6 to be slipped into and out of the adaptive wrap top 10.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

What is claimed is:

1. A garment, comprising:
a supportive back portion configured to overlap an upper torso of a wearer's back;
a front portion configured to overlap a chest of the wearer;
an interfaced elastic bottom panel comprising an interfaced layer and elastic that maintains a place of the garment with respect to the wearer; and
a closure mechanism configured to regulate a pressure of the garment against the wearer's upper torso.

2. The garment of claim 1, wherein the supportive back portion comprises material that substantially covers and exerts pressure against an upper portion and a middle portion of the wearer's back to provide support and improve posture.

3. The garment of claim 1, where in the supportive back portion comprises of cotton, elastic, polyester, linen, rayon, any types of blends, including recycled, any other fabrics out of natural vegetable and man-made fibers, or any other materials used by those skilled in the art.

4. The garment of claim 1, where in the interfaced supportive side panels comprises of plastic, cotton, cotton-polyester blends, and any other fabric that is made out of man-made fibers as well as vegetable natural fibers, including recycled materials, or any other materials commonly used by those skilled in the art.

5. The garment of claim 1, wherein the interfaced elastic bottom panel is configured with an anti-slip strip or coating.

6. The garment of claim 1, wherein the closure mechanism comprises a series of hook and loop fasteners.

7. The garment of claim 1, wherein the closure mechanism comprises a series of press stud fasteners or snaps.

8. The garment of claim 1, wherein the closure mechanism comprises a series of hook and eye fasteners.

9. The garment of claim 1, wherein the garment is configured to be worn as an active wear wrap top.

10. The garment of claim 1, wherein the garment is configured to be worn as a brasserie.

11. The garment of claim 1, wherein the front portion further comprises two slits and corresponding pockets, each slit and pocket configured to receive a removable bra pad.

12. The garment of claim 1, wherein two holes are provided on opposite sides of the front portion and supportive back portion which are configured to receive arms of the wearer.

13. The garment of claim 1, further comprising:
a first finishing hem comprising elastic and stitched to a perimeter of the front portion.

14. The garment of claim 1, further comprising:
a first finishing hem comprising elastic with anti-slip strip or coating and stitched to a perimeter of the front portion.

15. The garment of claim 1, further comprising:
a second finishing hem comprising elastic with an anti-slip strip or coating and stitched to a perimeter of the fused bottom panel.

16. The garment of claim 1, wherein the front portion comprises a double layer of material.

17. The garment of claim 1, wherein the supportive back portion comprises a posture corrective material comprising a double layered material in various shapes.

18. The garment of claim 1, wherein the supportive back portion comprises an additional supportive cross feature comprising of a first back panel, a secondary material interfaced from the upper right portion to the lower left portion, and a third material interfaced from the upper left portion to the lower right portion.
